(19) United States
(12) Patent Application Publication
(10) Pub. No.: US 2009/0111182 A1
(43) Pub. Date: Apr. 30, 2009

(54) Method for Propagation of Plant

(75) Inventors: Masaki Momose, Tochigi (JP); Yukiko Yamazaki, Tochigi (JP); Hiroshi Okawa, Tokyo (JP); Kanji Mamiya, Tochigi (JP); Noboru Onishi, Tochigi (JP)

Correspondence Address: FOLEY AND LARDNER LLP;SUITE 500, 3000 K STREET NW, WASHINGTON, DC, 20007, US

(73) Assignee: KIRIN HOLDINGS KABUSHIKI KAISHA

(21) Appl. No.: 12/085,929

(22) PCT Filed: Nov. 30, 2006
(86) PCT No.: PCT/JP2006/324390

§ 371 Date: May 30, 2008

(30) Foreign Application Priority Data
Dec. 1, 2005 (JP) 2005-348163

(51) Int. Cl. C12N 5/04

(52) U.S. Cl. 435/430.1

(57) ABSTRACT

A novel method is provided, which makes it possible to perform mass propagation of a plant of the family Araceae, the family Bromeliaceae, or the family Marantaceae. Specifically, a method for producing an embryogenic callus, comprising culturing a leaf sheath or a part thereof of a plant selected from the group consisting of the family Araceae, the family Bromeliaceae, and the family Marantaceae as an explant and then inducing an embryogenic callus and a propagation method comprising regenerating a plant through induction of a somatic embryo from the embryogenic callus obtained by the above method are also provided herein.

### TECHNICAL FIELD

The present invention relates to a method for propagation of plants. In particular, the present invention relates to a method for propagation of plants of the family Araceae, the family Bromeliaceae, and the family Marantaceae.

### BACKGROUND ART

Many plants important for both edible use and ornamental purposes belong to the family Araceae, the family Bromeliaceae, and the family Marantaceae. Various methods including cutting, division, use of tubers, and the like are used for propagation of these plants. Particularly for ornamental plants, propagation by a tissue culture method has been broadly spread and established. A currently employed tissue culture method is a method that involves inducing plantlets with multiple shoots on solid medium (medium solidified with agar, Gelrite, or the like).

However, conventional tissue culture methods are problematic in that the resulting propagation rates are low and mass propagation is difficult without the use of manual techniques. Furthermore, the work for propagation involves cutting shoots (contained by plantlets with multiple shoots) one by one and then transplanting the shoots one by one onto solid medium, so that the resulting working efficiency is low. Because of these reasons, the cost for production of these plants seedling is high. Furthermore, because of such low propagation rate, dissemination of new cultivars takes long time periods. They are limiting factors in terms of improvement and supply of cultivars that answer market needs.

One technique for elevating the propagation rate under culture conditions is a propagation method using somatic embryos, by which embryogenic callus is induced from plant tissues and then somatic embryos are generated from the embryogenic callus. Many studies have been conducted using this technique on carrot (the family Umbelliferae) as a typical example, with other examples including alfalfa (the family Leguminosae), celery (the family Umbelliferae), rice (the family Poaceae (Gramineae)), asparagus (the family Liliaceae), sweet potato (the family Convolvulaceae), and sugarcane (the family Poaceae (Gramineae)). In some plant types such as carrot, celery, and rice, acquisition of high production efficiencies for producing somatic embryos from embryogenic callus has been achieved. Thus this technique is increasingly examined as a mass propagation technique. Several tens of thousands of somatic embryos have been obtained from 1 g of embryogenic callus in some cases (for example, see Plant Cell, Tissue and Organ Culture 39, p 137, 1994).

However, conventional propagation methods using somatic embryos are problematic in that somatic embryo induction efficiencies sufficient for use in mass propagation have not been obtained in many cases regarding plants other than some of the aforementioned plants. In particular, the number of cases in which somatic embryo induction has been examined for the family Araceae, the family Bromeliaceae, or the family Marantaceae is very small. Furthermore, no technology leading to mass propagation has ever been established.

For example, somatic embryo induction from filaments has been confirmed for Spathiphyllum, which is an important ornamental plant belonging to the family Araceae (for example, see Acta Horticulturae 520, p 263, 2000). However, the test has only been used for gene recombination or mutation. This technique is not a method that involves embryogenic callus induction for propagation, but rather it involves direct induction of somatic embryos from filaments. Thus, the technique is problematic in that the propagation rate achieved with the use of a single explant is extremely limited. Moreover, somatic embryos are induced without being separated from anthers in liquid medium, so that such conditions of the somatic embryos have been suggested to be undesirable for the subsequent plant regeneration. In addition, the technique is also inappropriate in that when filaments are used as explants, the timing for collecting the filaments is limited to flowering time, and the quantity of the filaments as initial materials is limited for the purpose of propagation.

Furthermore, there has been a case in which somatic embryos were induced in Anthurium plants other than Spathiphyllum of the family Araceae (for example, see Plant Cell, Tissue and Organ Culture 48, p 189, 1997). Leaf sections are thought to be the most adequate for callus induction. Callus propagation from leaf sections and formation of somatic embryos from the calli have been reported. Furthermore, in Colocasia, callus induction from the cortical layers of tubers and redifferentiation that may take place via somatic embryos have been reported (for example, see Kyushu Agricultural Research, No. 54, p 196, 1992). Moreover, there has been a case in which embryogenic callus was induced in sugarcane of the family Poaceae (Gramineae) using leaf sheaths as explants (for example, see Protoplasma 118, p 169, 1983).

However, all of these reports lack descriptions concerning efficiencies of inducing somatic embryos from embryogenic callus and descriptions concerning efficiencies of redifferentiation of somatic embryos into plants. Moreover, these techniques reported are problematic in that somatic embryo induction is performed using solid medium alone, and thus they are not practical techniques. Furthermore, in the case of sugarcane leaf sheaths used as explants, the efficiency of inducing embryogenic callus is low when a leaf sheath part within 1 cm from the growing point is used. Hence, a part 1 cm to 2 cm away from the growing point is thought to be optimum.

Meanwhile, no reports have been made concerning somatic embryo induction in Guzmania of the family Bromeliaceae or in Calathea of the family Marantaceae, which are also important ornamental plants similar to Spathiphyllum. Accordingly, it is concluded that the use of somatic embryos for mass propagation of these plant species is extremely difficult. Hence, it is thought that almost no examination has been currently conducted for these plant species.

### DISCLOSURE OF THE INVENTION

An object of the present invention is to provide a novel method that enables mass propagation of plants of the family Araceae, the family Bromeliaceae, and the family Marantaceae.

As a result of intensive studies, the present inventors have discovered a method that surprisingly enables production of callus, wherein the method has high efficiency of inducing callus and the calli include embryogenic callus at high frequencies, through the use of the leaf sheaths of the plants of the family Araceae, the family Bromeliaceae, and the family Marantaceae as explants.

Specifically, in one aspect of the present invention, a method for producing an embryogenic callus is provided, which comprises culturing a leaf sheath or a part thereof of a plant selected from the group consisting of the family Araceae, the family Bromeliaceae, and the family Marantaceae as an explant and then inducing an embryogenic callus.

In an embodiment thereof, the explant is itself a leaf sheath, and in particular a leaf sheath base or a part near the leaf sheath base.

In another embodiment thereof, the explant is a part of a leaf sheath base, which is removed from a stem. Here the “leaf sheath base” refers to the lower ¼ part of a long leaf sheath section and the “part near the leaf sheath base” refers to the lower ½ part of such a long leaf sheath section from which the leaf sheath base has been removed.

In another aspect of the present invention, a propagation method for plants of the family Araceae, the family Bromeliaceae, and the family Marantaceae is provided, which comprises regenerating plants through induction of somatic embryos from the embryogenic callus that has been induced from leaf sheaths or parts thereof as explants of the plants.

In an embodiment thereof, the method of the present invention comprises the steps of culturing a plant leaf sheath or a part thereof as an explant to induce an embryogenic callus, inducing a somatic embryo from the embryogenic callus, and then causing germination and rooting of the somatic embryo.

In an embodiment thereof, the explant is a leaf sheath base or a part near the leaf sheath base of a leaf sheath.

In an embodiment thereof, the explant is a part of the leaf sheath base, which has been separated from the stem.

In another embodiment thereof, medium to be used in the step of inducing a somatic embryo contains glutamic acid and/or proline.

In another embodiment thereof, medium to be used in the step of inducing a somatic embryo is liquid medium.

In further embodiment thereof, medium to be used in the step of inducing a somatic embryo contains fructose and/or 6-benzyladenine.

This description includes part or all of the contents as disclosed in the description and/or drawings of Japanese Patent Application No. 2005-348163, which is a priority document of the present application.

### BRIEF DESCRIPTION OF THE DRAWINGS

FIG. 1 shows the status of somatic embryos obtained from experimental group 2 (the group to which glutamic acid was added) and a control group in Example 2.

FIG. 2 shows the germination status of somatic embryos obtained from experimental group 2 (the group to which glutamic acid was added) and a control group in Example 2.

### BEST MODE FOR CARRYING OUT THE INVENTION

### Definition

In the present invention, “embryogenic callus” refers to cream-colored granular dedifferentiated cells or tissues being capable of differentiating into plants via differentiating into somatic embryos, having soft forms (easily disintegrated when force is applied), and each having a diameter of less than 1 mm.

In the present invention, “somatic embryo” refers to an embryo that is generated from cultured plant somatic cells (tissue) and further develops into a normal plant.

In the present invention, “leaf sheath” refers to a base of a leaf, which is in a form that covers the stem like a sheath.

Step of Inducing Embryogenic Callus from Leaf Sheath Explant

First, a method for preparing explants from the leaf sheaths of plants of the family Araceae, the family Bromeliaceae, and the family Marantaceae is as described below.

It is not always required to collect leaf sheaths to be used in the present invention from cultured seedlings. Such leaf sheaths may also be collected from plants that are maintained under open conditions, such as in a greenhouse. In this case, leaf sheaths are subjected to surface sterilization according to a standard method, so as to prepare explants. When cultured seedlings are used, surface sterilization is not necessary. Any parts of leaf sheaths may be used, as long as they are of leaf sheaths. Preferably, leaf sheath parts that are located near the bases are used. Further preferably, when the base of a leaf, such as a leaf sheath, is collected from a plant seedling, for example, the leaf sheath is collected from the plant seedling, so that the leaf sheath is peeled off from the plant seedling without cutting it using a scalpel or the like, and then a part containing the thus removed (peeled) portion is used. Callus may also be obtained from leaves or roots; however, the frequency of such obtainment from leaves or roots is significantly lower than that of from leaf sheaths. Moreover, callus obtainment from leaves or roots is unfavorable since embryogenic calli cannot be easily induced in the subsequent step.

Next, a method for producing embryogenic callus by placing the thus prepared leaf sheath explants on medium is as described below.

First, leaf sheath explants obtained as described above are placed on callus induction medium, so that callus induction is performed. A basal medium that is used for callus induction is medium that is generally used for tissue culture, such as MS medium (Physiol. Plant, 15, p 143, 1962). One (1) % to 6% (the same weight/volume % is used below) and preferably 2% to 4% sucrose is used as a sugar source. As an auxin that is a plant growth regulating substance, preferably 1 ppm to 8 ppm and preferably 3 ppm to 5 ppm 2,4-dichlorophenoxyacetic acid (2,4-D) is used. As other auxins, indol-3-acetic acid (IAA), indole-3-butylic acid (IBA), 1-naphthaleneacetic acid (NAA), 4-chlorophenoxyacetic acid (CPA), chloromethylphenoxyacetic acid (MCPA), 2,4,5-trichlorophenoxyacetic acid (2,4,5-T), dichloromethoxybenzoic acid (DICAMBA), and trichloroaminopicolinic acid (PICLORAM) are used. As a cytokinin, 0.05 ppm to 2 ppm and preferably 0.1 ppm to 0.3 ppm 6-benzyladenine (BA) is preferably used. As other cytokinins, zeatin (ZEA), kinetin (KN), 6-(benzylamino)-9-(2-tetrahydropyranyl)-9H-purine (PBA), 2-isopentenyladenine (2ip), thidiazuron (TDZ), and the like are used. The most preferable result is obtained under conditions where 2,4-D and BA are used in combination. When 2,4-D is less than 1 ppm, plants are readily generated together with callus; and when 2,4-D exceeds 8 ppm, tissue easily undergoes browning. Furthermore, 50 ppm to 200 ppm casein acid hydrolysate and 0.1 mM to 10 mM MES as a buffer may also be added. The pH for medium ranges from 5 to 7. After pH adjustment, the medium is solidified using agar (0.8% to 1.2%) or Gelrite (0.1% to 0.3%). Containers to be used herein are not particularly limited, as long as they are used for plant tissue culture (e.g., a plant box produced by Asahi Techno Glass Corporation and internal volume of 300 ml). A dark place is employed for the light environment. The temperature ranges from 20° C. to 30° C. and preferably ranges from 23° C. to 27° C. The period for culturing ranges from 4 to 12 weeks and preferably ranges from 5 to 8 weeks.

Callus obtained as described above is subcultured on the same medium. Embryogenic callus that is generated therefrom is selected and then transplanted on the same medium, so that proliferation of the embryogenic callus alone is performed. Subsequently, with the use of similar operation, embryogenic callus is maintained and proliferated. The light and temperature conditions employed herein are the same as those employed for callus induction. The period for culturing ranges from 4 to 6 weeks.

For example, embryogenic calli (obtained by the above methods of the present invention in the following Examples) of the plants of the genus Spathiphyllum of the family Araceae, the genus Guzmania of the family Bromeliaceae, and the genus Calathea of the family Marantaceae are characterized by having cream-colored soft forms (easily disintegrated when force is applied) and each having a diameter of less than 1 mm.

Embryogenic callus obtained as described above can also be maintained as suspension-cultured cells in liquid medium with the same composition. In this case, medium is added in a volume accounting for approximately 15% to 30% of the flask volume to an about 300-ml to 500-ml Erlenmeyer flask.

Step of Inducing Somatic Embryo from Embryogenic Callus

A method for inducing somatic embryos from embryogenic callus that has been maintained and proliferated on agar medium as described above is as described below.

Most reported cases concerning somatic embryo induction involve the use of solid medium or liquid medium using small vessels such as Erlenmeyer flasks. Few cases concern mass propagation using fermenters (liquid medium). There has been a case in which somatic embryos obtained with the use of a fermenter were reported to be inferior to somatic embryos obtained with the use of solid medium in terms of the subsequent growth (Scale-Up and Automation in Plant Propagation p 35, 1991, Academic Press, Inc). Furthermore, there has been another case in which large-sized Erlenmeyer flasks were used in order to scale up somatic embryo induction in liquid medium (Scale-Up and Automation in Plant Propagation p 75, 1991, Academic Press, Inc). Somatic embryo induction in the present invention may be performed on solid medium such as agar medium. For the purpose of mass propagation, somatic embryo induction is performed in liquid medium and is further preferably performed using containers appropriate for large-scale and mass culture, such as fermenters. There has been a case of carrot that was subjected to mass induction of somatic embryos using a fermenter (Plant Cell, Tissue and Organ Culture 39, p 137, 1994), which is referable in the present invention. For the present invention, many commercially available fermenters for plant tissue culture (for example, produced by SIBATA SCIENTIFIC TECHNOLOGY LTD) can be used. An airlift fermenter may be used and preferably a fermenter of a type provided internally with agitation wings can be used. In the present invention, embryogenic callus is used as placement materials for somatic embryo induction that is performed directly in a fermenter without proliferation of suspension-cultured cells. Thus, the steps can be simplified and large amounts of somatic embryo can be induced from extremely small amounts of embryogenic callus.

Medium for somatic embryo induction is prepared using MS medium or the like as a basal medium, to which glutamic acid and/or proline is added at 1 mM to 10 mM and preferably 2 mM to 5 mM. Through addition of these amino acids, germination in the medium for somatic embryo induction can be suppressed and the quality and induction efficiency of somatic embryos can be enhanced. Basal medium is supplemented with 0.5% to 4% and preferably 1% to 2% sucrose as a sugar source. Depending on a plant cultivar to be cultured, 0.5% to 4% and preferably 0.5% to 2% fructose is added. Addition of fructose makes it possible to induce the somatic embryo with high efficiency in a cultivar in which it is difficult to induce the somatic embryo under general conditions, such as Spathiphyllum ‘Double Take’ used in Example 5. Furthermore, 1% to 6% and preferably 2% to 4% sorbitol or mannitol may be added as another sugar. As an auxin that is a plant growth regulating substance, 0 ppm to 2 ppm and preferably 0.01 ppm to 0.05 ppm 2,4-dichlorophenoxy acetic acid (2,4-D) can be preferably added. As other auxins, indol-3-acetic acid (IAA), indole-3-butylic acid (IBA), 1-naphthaleneacetic acid (NAA), 4-chlorophenoxyacetic acid (CPA), chloromethylphenoxyacetic acid (MCPA), 2,4,5-trichlorophenoxyacetic acid (2,4,5-T), dichloromethoxybenzoic acid (DICAMBA), and trichloroaminopicolinic acid (PICLORAM) can be added. As a cytokinin, 0 ppm to 0.5 ppm and preferably 0.05 ppm to 0.2 ppm 6-benzyladenine (BA) can be preferably added. Through addition of BA, differences in somatic embryo induction efficiency between cultivars can be reduced. As other cytokinins, zeatin (ZEA), kinetin (KN), 6-(benzylamino)-9-(2-tetrahydropyranyl)-9H-purine (PBA), 2-isopentenyladenine (2ip), thidiazuron (TDZ), and the like are used. Moreover, 0.1 mM to 10 mM MES may also be added as a buffer. The pH for medium ranges from 5 to 7.

Regarding the light environment, light conditions (a day length ranging from 12 to 16 hours and photosynthetic photon flux density ranging from 5.7 μmole/m²/sec to 34.2 μmole/m²/sec) are preferred. Dark conditions may be employed herein. The temperature ranges from 20° C. to 30° C. and desirably ranges from 23° C. to 27° C. The period for culturing ranges from 4 to 12 weeks and preferably ranges from 5 to 10 weeks.

As materials to be placed in a fermenter, both embryogenic callus that has been maintained and proliferated on solid medium and suspension-cultured cells that have been maintained and proliferated in liquid medium may be used. The somatic embryo induction efficiency achieved in the present invention using liquid medium from embryogenic callus (maintained on solid medium) is extremely high. Hence, such embryogenic callus is desirably used as materials for placement. When suspension-cultured cells are used, instruments such as a flask shaker and various tools required for collection of suspension-cultured cells will be required. Hence, the use of suspension-cultured cells is not optimum for mass culture. Embryogenic callus induced in the present invention is characterized by extremely high somatic embryo induction efficiency in liquid medium as described above. Thus, the amount of embryogenic cell to be placed per unit medium may be a small amount and specifically ranges from approximately 0.05 g/l to 5 g/l and preferably ranges from 0.1 g/l to 2 g/l.

### Step for Dehydration of Somatic Embryo

Collected somatic embryos have their own germination ability on solid medium, but the germination percentage is low. Hence, the thus redifferentiated individual plants tend to be in a status referred to as vitrification. When somatic embryos are dehydrated, the redifferentiation efficiency is improved and seedlings become normal. Containers to be used for dehydration are not particularly limited. When such a container is used for treatment of a small amount of somatic embryo, a petri dish (e.g., a diameter of 9 cm and height of 1.5 cm) is used. When such a container is used for treatment of a large amount of somatic embryo, a box-type container made of transparent plastic (for example, a size of approximately 22 cm×17 cm×7 cm) is used. In both cases, a paper towel is placed on the bottom and then an adequate amount of somatic embryo is added (in the case of a 9-cm petri dish, approximately 1 g to 20 g; in the case of the above box-type container, approximately 30 g to 100 g). Regarding the light environment, preferred light conditions comprise a day length ranging from 12 to 16 hours and photosynthetic photon flux density ranging from 1.1 μmole/m²/sec to 34.2 μmole/m²/sec and preferably ranging from 3.4 μmole/m²/sec to 22.8 μmole/m²/sec. Dark conditions may also be employed herein. The temperature ranges from 20° C. to 30° C. and preferably ranges from 23° C. to 27° C. The period ranges from 3 days to 21 days and preferably ranges from 5 days to 14 days. Dehydration is performed so that the fresh weight of somatic embryo accounts for 40% to 80% and preferably 50% to 70% of the weight of the same measured before the start of dehydration. Dehydrated somatic embryo can be stored at normal temperature for 3 to 12 months by placing 10 g to 20 g of the embryo in a 9-cm petri dish in which a paper towel is placed on the bottom. The preferable storage environment may be identical to the above environment for dehydration, but is not limited thereto. Dehydrated somatic embryos can also be stored at a low temperature in a dark place.

### Step of Causing the Germination of Somatic Embryo

Dehydrated somatic embryos germinate at high efficiency on both solid medium and liquid medium. However, the use of liquid medium is preferred for the purpose of mass propagation. Medium to be used for germination is prepared using MS medium or the like as basal medium and adding 1% to 6% and preferably 2% to 4% sucrose as a sugar source to the medium. When rooting or the like takes place aggressively, 1% to 6% and desirably 2% to 4% sorbitol or mannitol may be added as another sugar. Addition of a plant growth regulating substance is not particularly required, but auxins or cytokinins may be added to promote germination. The pH for medium ranges from 5 to 7. Regarding the light environment, light conditions comprise a day length ranging from 12 to 16 hours and photosynthetic photon flux density ranging from 1.1 μmole/m²/sec to 34.2 μmole/m²/sec and preferably ranging from 2.3 μmole/m²/sec to 22.8 μmole/m²/sec. The temperature ranges from 20° C. to 30° C. and preferably ranges from 23° C. to 27° C. In the case of solid medium, medium is solidified using agar (0.8% to 1.2%) or Gelrite (0.1% to 0.3%). Containers to be used herein are not particularly limited, as long as they are used for plant tissue culture. In the case of solid medium, the above described plant box is used. In the case of liquid medium, the above described agitation or airlift fermenter is used. Regarding the amount of dehydrated somatic embryo to be placed, in the case of solid medium, 50 ml of medium is added to the above plant box and then 0.05 g to 1 g and preferably 0.1 g to 0.5 g of somatic embryo is placed. In the case of liquid medium, 0.1 g/l to 2 g/l and desirably 0.3 g/l to 1 g/l of somatic embryo is placed. The period for culturing ranges from, in the case of solid medium, 4 to 12 weeks and in the case of liquid medium, ranges from 3 to 6 weeks. During this period, dehydrated somatic embryos undergo rooting and develop into plant bodies of a size such that several leaves have developed. In liquid medium, leaf development of a plant is more suppressed in liquid medium due to the effect of agitation than that in solid medium. A plant that is induced in such liquid medium can be directly acclimatized in a greenhouse or the like. However, such a plant can also be transplanted on solid medium and then further grown. Conditions for culture on solid medium in such case are identical to the above germination conditions for dehydrated somatic embryos. The step of the culture may also be performed by static culture using liquid medium. Conditions to be employed for this case may be identical to conditions for somatic embryo germination on solid medium except that the use of a solidification agent (agar or Gelrite) is excluded from the above conditions for the culture on solid medium. Accordingly, through the use of a fermenter, germination and regeneration of plant bodies from somatic embryos can be efficiently performed. In addition, there have been no cases before the present invention in which germination from somatic embryos and regeneration of plants have been performed efficiently using a fermenter according to a conventional propagation method using somatic embryos.

Transplantation into Greenhouse

Plants that have redifferentiated from somatic embryos grow normally in a greenhouse. Soil to be used for transplantation is not particularly limited. Culture soil that is commercially available for raising of seedlings may be used herein. After transplantation of plants, moderate humidification and light shielding are preferably performed for approximately 1 to 3 weeks.

### Applicable Plant

Plants that can be propagated by the above described techniques of the present invention are various plants belonging to the family Araceae, the family Bromeliaceae, and the family Marantaceae. Specific examples of plants belonging to the family Araceae include Colocasia, dasheen (Colocasia esculenta), konjak (Amorphophalus konjak), Spathiphyllum, Anthurium, Caladium, Aglaonema, Alocasia, Dieffenbachia, Monstera, Philodendron, pothos (Epipremnum aureum), Syngonium, calla, white arum (Lysichiton camtschatcense), skunk cabbage (Symplocarpus foetidus), calamus (Acorus calamus), and Pinellia ternate. Specific examples of plants belonging to the family Bromeliaceae include Neoregelia, Aechmea, Vriesea, Tillandsia, Cryptanthus, Ananas (pineapple), and Guzmania. Specific examples of plants belonging to the family Marantaceae include Maranta, Ctenanthe, Stromanthe, and Calathea.

### EXAMPLE 1

The present invention is further illustrated in detail with reference to the following examples. However, these examples do not limit the scope of the present invention.

Callus induction from various tissues of the cultured seedlings of Spathiphyllum (cultivar Petite) and embryogenic callus induction from the aforementioned callus were performed. The cultured seedlings had been maintained with the subculturing thereof on solid medium prepared by adding 3% sucrose and 0.8% agar (Wako Pure Chemical Industries, Ltd., Tokyo, Japan) to MS medium under conditions of a pH adjusted to 5.8, a light place (photosynthetic photon flux density of 5.7 μmole/m²/sec and a day length of 16 hours), and 25° C. Plant boxes (produced by Asahi Techno Glass Corporation) were used as culture containers (internal volume 300 ml). On week 4 of culturing, leaf sheaths were collected from the cultured seedlings such that the leaf sheaths were peeled off from the strains. Roughly-2-cm sections (leaf sheath bases) containing peeled portions were used as explants. The explants were placed at 6 sections per container on MS medium (pH 5.8; hereinafter, “callus induction•proliferation medium”) prepared by adding 3% sucrose, 4 ppm 2,4-D, 0.2 ppm BA, 100 ppm casein acid hydrolysate, and 5 mM MES and then solidifying the medium using 0.8% agar (containers used herein were plant boxes and the amount of each medium was 50 ml). A total of 4 containers were used. The explants were cultured in a dark place and under conditions of 25° C. for 8 weeks and then the callus induction state was examined. As a result, calli were induced in peeled portions among 92% of placed sections (the ratio of the number of sections from which calli had been induced to the number of sections placed=22:24). These calli were subcultured at 0.2 g/container on the same medium and then cultured for 1 month, so that embryogenic callus was selected. Such embryogenic callus was induced with an induction percentage of 50% (the ratio of the number of containers in which embryogenic callus had been induced to the number of containers used for treatment=4:8).

The embryogenic callus (0.05 g each) was placed in a 500-ml Erlenmeyer flask supplemented with 160 ml of MS liquid medium (pH 5.8) containing 1% sucrose, 3% sorbitol, and 5 mM MES and then cultured with shaking (80 rpm) at 25° C. in a light place (a day length of 16 hours and photosynthetic photon flux density of 22.8 μmole/m²/sec) for 6 weeks. Thus, somatic embryos were formed. These somatic embryos were placed at 0.2 g each per plant box supplemented with 50 ml of MS medium containing 3% sucrose (solidified using 0.8% agar, pH 5.8, hereinafter, “germination medium”) and then cultured at 25° C. in a light place (a day length of 16 hours and photosynthetic photon flux density of 5.7 μmole/m²/sec) for 8 weeks. The somatic embryos had germinated so that complete plants could be obtained. The embryogenic callus could be maintained for 2 years while retaining their ability of inducing somatic embryos by subculturing them every 4 weeks into “callus induction•proliferation medium.”

### COMPARATIVE EXAMPLE 1

Roots and leaves that are relatively generally used were used as explants for callus induction instead of leaf sheaths of the cultured seedlings of Spathiphyllum (cultivar Petite). Roughly-1-cm root sections were prepared by removing growing points from roots. Leaf sections of approximately 5 mm by 5 mm were prepared from leaves. These sections were placed under the same conditions as those of Example 1. Eight (8) weeks later, the callus induction state was examined. Almost no callus was formed from the leaves. Moreover, callus was partially formed from the roots, but the maintenance thereof in the form of callus was difficult because of the generation of adventitious roots. Therefore, ultimately no somatic embryos could be obtained from any explants.

### EXAMPLE 2

The effects of proline and glutamic acid on somatic embryo induction were examined using embryogenic callus of the cultivar Petite, which had been induced under the same conditions as those of Example 1. MS liquid medium (pH 5.8) containing 1% sucrose, 3% sorbitol, and MES 5 mM was used as basal medium. Proline and glutamic acid were added to the media at the following concentrations. Subsequently, 0.05 g each of the embryogenic callus was placed in a 300-ml Erlenmeyer flask (5 flasks per group) containing 40 ml of each medium and then cultured with shaking (70 rpm) for 6 weeks at 25° C. in a light place (a day length of 16 hours and photosynthetic photon flux density of 22.8 μmole/m²/sec). The fresh weights the thus induced somatic embryos were measured (Table 1).

TABLE 1
Effects of proline and glutamic acid on somatic embryo induction

| | Proline | Glutamic | Amount of embryo |
|---|---|---|---|
| Group | (mM) | acid (mM) | collected (g/flask) |
| Control group | 0 | 0 | 2.1 |
| Experimental group 1 | 3 | 0 | 3.4 |
| Experimental group 2 | 0 | 3 | 3.5 |
| Experimental group 3 | 3 | 3 | 3.9 |
| Experimental group 4 | 5 | 5 | 3.7 |
| Experimental group 5 | 10 | 10 | 4.4 |

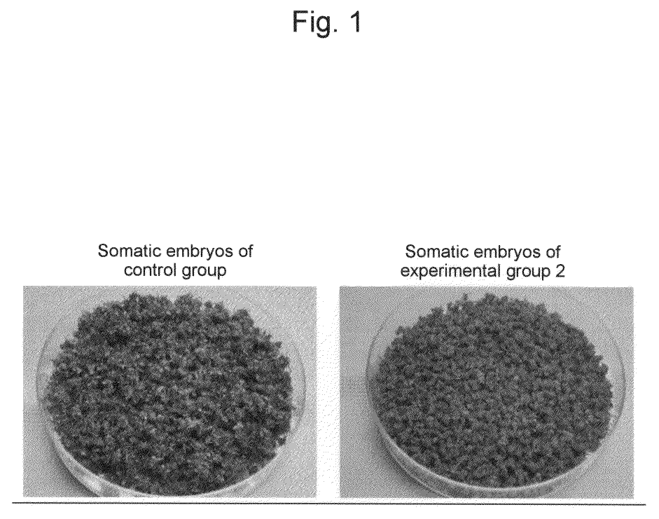

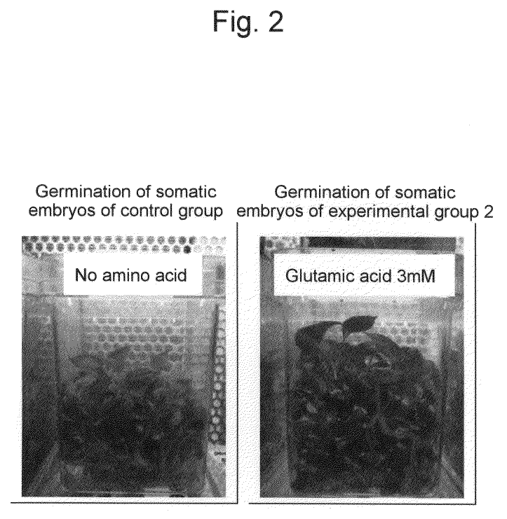

The weights of embryo collected in groups to which proline and/or glutamic acid had been added were each significantly greater than that of the control group. Moreover, in these groups, the weight of embryos collected was increased and somatic embryo germination in liquid medium were suppressed, so that no tissue portions that had undergone browning were observed and uniform somatic embryos were obtained (FIG. 1). Furthermore, the germination status of somatic embryos collected was observed to be more active than that in the control group and the degrees of chloroplast development in the groups were higher than that in the control group (FIG. 2). Based on these results, the effects of proline and/or glutamic acid added on the somatic embryo induction media were evident.

### EXAMPLE 3

Somatic embryo induction was performed in a fermenter using embryogenic callus of the cultivar Petite, which had been induced under the same conditions as those of Example 1. As medium for somatic embryo induction, MS medium supplemented with 1% sucrose, 3% sorbitol, 0.02 ppm 2,4-D, 3 mM proline, 3 mM glutamic acid, and 5 mM MES (pH 5.8; hereinafter, “somatic embryo induction medium”) was used. As a fermenter, a cylindrical fermenter (internal volume of 8 l; hereinafter, “somatic embryo induction fermenter”) having agitation wings with a width of 15 cm (produced by SIBATA SCIENTIFIC TECHNOLOGY LTD), a bottom area of 314 cm², and a height of 24 cm was used. Two (2) g each of embryogenic callus was placed in a fermenter (a total of 9 fermenters) containing 5 l of somatic embryo induction medium. Somatic embryo induction was performed by rotating the agitation wings at a rate of 50 rpm (per minute) while performing aeration (0.06 vvm) from the bottom. Culturing was performed under conditions of 25° C. in a light place (a day length of 16 hours and photosynthetic photon flux density of 22.8 μmole/m²/sec) for 5 weeks. Somatic embryos were then collected using a stainless strainer. The thus collected somatic embryos were each of a 2-mm to 3-mm size and in a uniform shape with suppressed elongation of shoots and roots. Excess water of these somatic embryos was adsorbed using paper towels and then 60 g of the embryos was placed in each box-type container made of transparent plastic (with a size of 22 cm×17 cm×7 cm, a paper towel was placed on the bottom). The average fresh weight of the collected somatic embryos was 352 g per fermenter. The average fresh weight measured after 1 week of the dehydration step was 237 g accounting for 67% of the fresh weight measured before dehydration. The dehydrated somatic embryos (0.5 g each) were placed in a plant box to which 50 ml of germination medium had been dispensed. The embryos were cultured for 10 weeks under light conditions (a day length of 16 hours and photosynthetic photon flux density of 5.7 μmole/m²/sec). Thus, approximately 50 complete redifferentiated plants were obtained on average per container. This was high propagation efficiency equivalent to 100 seedlings per g of dehydrated somatic embryo, 24,000 seedlings per somatic embryo induction fermenter, and 12,000 seedlings per g of embryogenic callus.

### COMPARATIVE EXAMPLE 2

The medium of the control group in Example 2 was used as medium for a somatic embryo induction fermenter. The average fresh weight of somatic embryos collected was 176 g (the number of fermenters=10) per fermenter and the average weight measured after dehydration was 104 g, accounting for 50% and 44%, respectively, of the results in Example 3.

### EXAMPLE 4

The dehydrated somatic embryos (3.8 g each) obtained from Example 3 were planted in a cylindrical culture container (bottom area of approximately 190 cm², a height of approximately 45 cm, and an internal volume of approximately 6 l; hereinafter, “plant induction fermenter”) containing 5 l of liquid medium (pH 5.8) prepared by adding 3% sucrose to MS medium. The somatic embryos were cultured while performing aeration (0.06 vvm) from the bottom at 25° C. in a light place (a day length of 16 hours and photosynthetic photon flux density of 22.8 μmole/m²/sec) for 6 weeks. The somatic embryos did not grow excessively while developing leaves and roots because of agitation applied thereto. Thus, compact and uniform plantlets with multiple shoots with diameters ranging from approximately 1 cm to 2 cm were formed, so that 125 g of such plantlet was obtained per container. These plantlets with multiple shoots were collected, divided one by one (one shoot by one shoot), placed in “germination medium,” and then cultured in a light place (a day length of 16 hours and photosynthetic photon flux density of 5.7 μmole/m²/sec) for 4 weeks. Thus, complete plants with developed and elongated leaves and roots were obtained. The number of seedlings was 250 per g of dehydrated somatic embryo, 950 seedlings per plant induction fermenter, or 59,000 seedlings per somatic embryo induction fermenter. It was confirmed that production efficiency of seedlings from somatic embryos could be further elevated via the liquid germination step. Dehydrated somatic embryos that were from the same lot as that of the dehydrated somatic embryos tested herein could germinate and grow with efficiency similar to that achieved before storage even after 6 months of storage at normal temperature.

### EXAMPLE 5

Embryogenic callus was induced from Double Take, a cultivar of Spathiphyllum, by the same techniques as those of Example 1. Somatic embryo induction from the embryogenic callus was attempted using the “somatic embryo induction medium” of Example 3. The weights of collected somatic embryos in the case of the cultivar Double Take were lower than those in the case of the cultivar Petite. Furthermore, the somatic embryos obtained from the cultivar Double Take were revealed to more easily undergo browning and wither compared with those obtained from the cultivar Petite. Hence, the effects of alteration in the sugar composition of the “somatic embryo induction medium” and the addition of BA were examined. Media with compositions listed in Table 2 were prepared (compositions other than those listed in Table 2 were the same as those of the “somatic embryo induction medium.”). Culturing was performed under the same conditions as those of Example 2 and then the weights of collected somatic embryos were measured (Table 2).

TABLE 2
Effects of the sugar composition of somatic embryo induction
medium and addition of BA on somatic embryo induction

| | | | | | Weight of |
|---|---|---|---|---|---|
| | | | Fruc- | | somatic |
| | Sucrose | Sorbitol | tose | BA | embryo |
| Group | (%) | (%) | (%) | (ppm) | (g)/flask |
| Control group | 1 | 3 | 0 | 0 | 1.5 |
| Experimental group 1 | 0 | 3 | 1.1 | 0 | 3.8 |
| Experimental group 2 | 0 | 3 | 1.1 | 0.1 | 4.9 |
| Experimental group 3 | 1 | 0 | 1.1 | 0.1 | 6.1 |

Somatic embryo induction percentages were greatly increased in the groups to which fructose and BA had been added, compared with the control group. Furthermore, comparison was made using solid medium in terms of the efficiency of plant regeneration from the thus obtained somatic embryos. The collected somatic embryos were added into a 9 cm×1.5 cm petri dish and then dehydrated under the conditions of Example 3. Such dehydrated somatic embryos (0.2 g) were placed in a plant box to which 50 ml of “germination medium” had been dispensed. Culturing was performed under conditions of 25° C. in a light place (a day length of 16 hours and photosynthetic photon flux density of 5.7 μmole/m²/sec) for 6 weeks. As a result, whereas the number of plants obtained per g of the somatic embryo was 47 in the control group, the same was 88 in the experimental group 2. Therefore, it could be confirmed in the groups cultured under conditions involving the addition of fructose that such conditions are effective for the reduction of differences between cultivars in terms of efficiency for somatic embryo induction and improvement in the efficiency of plant regeneration from somatic embryos.

Furthermore, the effects of alteration of the compositions of the above media were examined using the “somatic embryo induction fermenter.” Somatic embryo induction was performed using the same conditions as those of Example 3, except that the medium of the control group and the medium of the experimental group 3 were used. As a result, whereas the weight of the thus obtained somatic embryo (after dehydration) was 72 g in the group for which the control medium had been used, the weight of the same was 160 g in the group for which the medium of experimental group 3 had been used. It was confirmed that the improved medium was also effective for mass induction of somatic embryos using a fermenter. Furthermore, somatic embryos collected from the medium of experimental group 3 were subjected to induction of germination by the method of Example 4 using liquid medium (only the period for culturing was altered to 8 weeks). Finally, the number of seedlings was 212 per dehydrated somatic embryo, 795 per plant induction fermenter, and 34,000 per somatic embryo induction fermenter, resulting in high-level effects of plant propagation.

### EXAMPLE 6

The techniques for embryogenic callus induction of Example 1 were applied to Guzmania (cultivar Anton). Callus was induced from leaf sheath bases with an induction percentage of 91% (the ratio of the number of sections from which callus had been induced to the number of sections placed=49:54). These calli were embryogenic calli having characteristics similar to those of Spathiphyllum. The conditions in Example 3 for somatic embryo induction using a fermenter were applied to the embryogenic calli. Fifty (50) g of the dehydrated somatic embryos were obtained from 2 g of the embryogenic callus placed. The thus obtained somatic embryos were placed on the “germination medium” of Example 3 and then cultured under the same conditions. Thus, plantlets with multiple shoots capable of uniformly and actively proliferating could be obtained. These plantlets with multiple shoots were divided one by one and then grown on the “germination medium” so that complete plants were generated. Finally, seedlings were obtained in an amount equivalent to 800,000 seedlings per somatic embryo induction fermenter. It was confirmed that the techniques of the present invention were also effective for Guzmania.

### EXAMPLE 7

The techniques for embryogenic callus induction of Example 1 were applied to Calathea (cultivar Picta Royale). Thus, callus was induced from leaf sheath bases with an induction percentage of 21% (the ratio of the number of sections from which callus had been induced to the number of placed sections=3:14). These calli were embryogenic calli having characteristics similar to those of Spathiphyllum and Guzmania. Such embryogenic callus (0.05 g) was placed in liquid medium (160 ml of the medium/500-ml Erlenmeyer flask) in which the concentration of 2,4-D in the somatic embryo induction medium of Example 3 had been altered to 0.2 ppm, and then it was cultured with shaking (70 rpm) at 25° C. in a light place (a day length of 16 hours and photosynthetic photon flux density of 22.8 μmole/m²/sec) for 10 weeks. Thus, 9.1 g of the somatic embryo was obtained on average per flask. These somatic embryos were dehydrated under the conditions of Example 5. Subsequently, 0.1 g each of the somatic embryo (dehydrated somatic embryo) was placed in a plant box containing 50 ml of “germination medium.” Culturing was performed under conditions of 25° C. in a light place (a day length of 16 hours and photosynthetic photon flux density of 5.7 μmole/m²/sec) for 12 weeks. As a result, 10 plants were observed to be regenerated on average per plant box (that is, the number of plants regenerated per g of embryogenic callus was 18,000). It was confirmed that the techniques of the present invention were also effective for Calathea.

### INDUSTRIAL APPLICABILITY

According to the method for producing an embryogenic callus of the present invention, a callus with extremely high efficiency for somatic embryo induction can be produced by culturing a leaf sheath or a part thereof as an explant.

According to the method for propagation of a plant of the present invention, propagation rate and working efficiency can be enhanced, and thus plant seedlings can be produced with higher efficiency at lower cost compared with conventional methods through improvement of a mass propagation method using somatic embryos. More specifically, calli (embryogenic calli) with extremely high efficiency for somatic embryo induction are induced from the leaf sheaths, leaf sheath bases, or parts near the bases of plants of the family Araceae, the family Bromeliaceae, the family Marantaceae, or the like, which have almost never been propagated from somatic embryos. Somatic embryos are then induced in large amounts in a fermenter appropriate for mass culture, such as a fermenter to which liquid medium has been added, and then caused to regenerate into plants on solid medium or in liquid medium. Therefore, plants can be rapidly propagated within a short period at low cost.

Moreover, measures to solve the problems that arise when a method using somatic embryos is applied for actual production are included herein. Examples of such measures include simplification of a step via the direct use of an embryogenic callus as a placement material for somatic embryo induction in a fermenter without proliferation of suspension-cultured cells, reduction of the difference between cultivars via improvement of somatic embryo induction medium, several months of storage of induced somatic embryos at normal temperature, and the like. According to the method of the present invention, it is made possible to perform short-period and low-cost mass propagation of various plants (and in particular, plants of the family Araceae, the family Bromeliaceae, and the family Marantaceae), the propagation of which has depended on conventional technology such as tissue culture methods and division methods.

The present invention can be used for mass propagation of plants of the family Araceae, the family Bromeliaceae, and the family Marantaceae.

All publications, patents, and patent applications cited in this description are herein incorporated by reference in their entirety.

1. A method for producing an embryogenic callus, comprising culturing a leaf sheath or a part thereof of a plant selected from the group consisting of the family Araceae, the family Bromeliaceae, and the family Marantaceae as an explant and then inducing an embryogenic callus.

2. The method according to claim 1, in which the explant is a leaf sheath base or a part near the leaf sheath base.

3. A method for propagation of a plant of the family Araceae, the family Bromeliaceae, or the family Marantaceae, comprising regenerating a plant through induction of a somatic embryo from an embryogenic callus that has been induced from a leaf sheath or a part thereof of the plant as an explant.
